(19) United States
(12) Patent Application Publication
(10) Pub. No.: US 2007/0259930 A1
(43) Pub. Date: Nov. 8, 2007

(54) COMPOSITIONS AND METHODS OF USING R(+) PRAMIPEXOLE

(75) Inventors: Michael E. Bozik, Pittsburgh, PA (US); Gregory T. Hebrank, Greensburg, PA (US); Thomas Petzinger JR., Pittsburgh, PA (US)

Correspondence Address: PEPPER HAMILTON LLP, ONE MELLON CENTER, 50TH FLOOR, 500 GRANT STREET, PITTSBURGH, PA, 15219, US

(73) Assignee: KNOPP NEUROSCIENCES, INC., Pittsburgh, PA (US)

(21) Appl. No.: 11/733,642

(22) Filed: Apr. 10, 2007

Related U.S. Application Data
(60) Provisional application No. 60/744,540, filed on Apr. 10, 2006.
(60) Provisional application No. 60/746,441, filed on May 4, 2006.
(60) Provisional application No. 60/747,317, filed on May 16, 2006.
(60) Provisional application No. 60/747,320, filed on May 16, 2006.
(60) Provisional application No. 60/829,066, filed on Oct. 11, 2006.
(60) Provisional application No. 60/870,009, filed on Dec. 14, 2006.
(60) Provisional application No. 60/894,799, filed on Mar. 14, 2007.
(60) Provisional application No. 60/894,829, filed on Mar. 14, 2007.
(60) Provisional application No. 60/894,835, filed on Mar. 14, 2007.

(51) Int. Cl. A61K 31/428

(52) U.S. Cl. 514/367

(57) ABSTRACT

Pharmaceutical compositions of R(+) pramipexole and methods of using such compositions for the treatment or prevention of diseases associated with or related to mitochondrial dysfunction or increased oxidative stress are disclosed.

### CROSS REFERENCE TO RELATED APPLICATIONS

This application claims the benefit of U.S. Provisional Application Ser. No. 60/744,540 entitled “R(+) Pramipexole for the Treatment of Age-Related Macular Degeneration” filed Apr. 10, 2006; U.S. Provisional Application Ser. No. 60/746,441 entitled “Tetrahydrobenzothiazoles and Uses Thereof” filed May 4, 2006; U.S. Provisional Application Ser. No. 60/747,317 entitled “Tetrahydrobenzothiazoles and Uses Thereof” filed May 16, 2006; U.S. Provisional Application Ser. No. 60/747,320 entitled “Tetrahydrobenzothiazoles and Uses Thereof” filed May 16, 2006; U.S. Provisional Application Ser. No. 60/829,066 entitled “Compositions and Methods of Treating and preventing Inflammatory Disorders” filed Oct. 11, 2006; U.S. Provisional Application Ser. No. 60/870,009 entitled “Compositions and Methods of Using R(+) Pramipexole”, filed Dec. 14, 2006; U.S. Provisional Application Ser. No. 60/894,799 entitled “Modified Release Formulations and Methods of Use of R(+) Pramipexole” filed Mar. 14, 2007; U.S. Provisional Application Ser. No. 60/894,829 entitled “Methods of Snythesizing and Purifying R(+) and S(−) Pramipexole” filed Mar. 14, 2007; and U.S. Provisional Application Ser. No. 60/894,835 entitled “Compositions and Methods of Using R(+) Pramipexole” filed Mar. 14, 2007; each of which is incorporated herein by reference in their entireties.

### BRIEF SUMMARY

Embodiments of the present invention related to methods of using or administering R(+) pramipexole for the treatment and/or prevention of diseases and conditions associated with or involving decreased mitochondrial function or mitochondrial dysfunction. Such diseases and conditions include, but are not limited to, age-related macular degeneration, type II diabetes, skin diseases and disorders, coronary and cardiovascular diseases and disorders, and inflammatory disorders.

Embodiments of the present invention related to methods of treating age-related macular degeneration comprising administering a therapeutically effective amount of R(+) pramipexole.

Further embodiments of the present invention relate to methods of treating of treating type II diabetes comprising administering a therapeutically effective amount of R(+) pramipexole.

Further embodiments of the present invention relate to methods of treating of treating skin disorders comprising administering a therapeutically effective amount of R(+) pramipexole.

Other embodiments of the present invention relate to methods of treating of treating cardiovascular disorders comprised of administering a therapeutically effective amount of R(+) pramipexole.

Further embodiments of the present invention relate to methods of treating of treating inflammatory disorders comprised of administering a therapeutically effective amount of R(+) pramipexole.

### BRIEF DESCRIPTION OF THE DRAWINGS—NOT APPLICABLE

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. All publications mentioned herein are incorporated by reference in their entirety.

It must also be noted that as used herein and in the appended claims, the singular forms “a”, “an”, and “the” include plural reference unless the context clearly dictates otherwise. Thus, for example, reference to a “salt” is a reference to one or more organic solvents and equivalents thereof known to those skilled in the art, and so forth.

As used herein, the term “about” means plus or minus 10% of the numerical value of the number with which it is being used. Therefore, about 50% means in the range of 45%-55%. Unless defined otherwise, all technical and scientific terms used herein have the same meanings as commonly understood by one of ordinary skill in the art.

As use herein, the terms “administration of” and or “administering” a compound should be understood to mean providing a compound of the invention or a prodrug of a compound of the invention to an individual in need of treatment. Within the scope of the use according to the invention pramipexole may be administered, for example, orally, transdermally, intrathecally, by inhalation or parenterally.

As used herein, the terms “enantiomers”, “stereoismers” and “optical isomers” may be used interchangeably, and refer to molecules which contain an asymmetric or chiral center and are mirror images of one another. Further, the terms “entantiomers”, “stereoisomers” or “optical isomers” describe a molecule which, in a given configuration, cannot be superimposed on its mirror image. As used herein, the term “optically pure” or “enantiomerically pure” may be taken to indicate that the compound contains at least 99.5% of a single optical isomer. The term “enantiomerically enriched” may be taken to indicate that at lest 51% of the material is a single optical isomer or enantiomer. The term “enantiomeric enrichment” as used herein refers to an increase in the amount of one enantiomer as compared to the other. A “racemic” mixture is a mixture of equal amounts of R(+) and S(−) enantiomers of a chiral molecule. Throughout this invention, the word “pramipexole” will refer to both the R(+) enantiomer and the S(−) enantiomer of pramipexole.

The term “pharmaceutical composition” shall mean a composition comprising at least one active ingredient, whereby the composition is amenable to investigation for a specified, efficacious outcome in a mammal (for example, without limitation, a human). Those or ordinary skill in the art will understand and appreciate the techniques appropriate for determining whether an active ingredient has a desired efficacious outcome based upon the needs of the artisan.

“Therapeutically effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reducing the severity of the pathology and/or symptomatology).

A “non-effective dose amount” as used herein refers to an amount of active compound or pharmaceutical agent that elicits a biological or medicinal response similar to the biological or medicinal response of a placebo as observed in a tissue, system, animal, individual or human that is being treated by a researcher, veterinarian, medical doctor or other clinician. A “non-effective dose amount” may therefore elicit no discernible difference from placebo in positive effects as observed in a tissue, system, animal, individual or human that is being treated by a researcher, veterinarian, medical doctor or other clinician. As such, the “non-effective dose amount” is not expected to (1) prevent a disease; for example, preventing a disease, condition or disorder in an individual that may be predisposed to the disease, condition or disorder but does not yet experience or display the pathology or symptomatology of the disease; (2) inhibit the disease; for example, inhibiting a disease, condition or disorder in an individual that is experiencing or displaying the pathology or symptomatology of the disease, condition or disorder (i.e., arresting further development of the pathology and/or symptomatology), or (3) ameliorate the disease; for example, ameliorating a disease, condition or disorder in an individual that is experiencing or displaying the pathology or symptomatology of the disease, condition or disorder (i.e., reversing the pathology and/or symptomatology).

An example involves S(−) pramipexole, the enantiomer of R(+) pramipexole. In monkeys treated with (1-methyl-4-phenyl-1,2,3,6-tetrahydropyridine), S(−) pramipexole has been shown to antagonize motor deficits and Parkinson-like symptoms in a dose-dependent manner, with the lowest effective oral dose being 0.053 mg/kg. This would be equivalent to a human dose of 0.017 mg/kg, or 1.2 mg for a 70 kg individual. In human trials, the lowest effective oral dose of S(−) pramipexole with a significant effect versus placebo in the treatment of Parkinson's disease was found to be 1.1 mg/day. Individual patients may need doses higher than 1.1 mg/kg to gain a sufficient effect above the placebo effect (Initial Scientific Discussion for the Approval of Mirapex from the European Agency for the Evaluation of Medicinal Products). In human trials, the lowest effective dose with a significant effect versus placebo in the treatment of restless legs syndrome was found to be 0.25 mg/day (Boehringer Ingelheim product insert for Mirapex®). Therefore, with reference to S(−) pramipexole, a non-effective dose amount may be an amount below 1.0 mg/day, below 0.75 mg/day, below 0.5 mg/day, below 0.25 mg/day, or preferably below 0.125 mg/day.

A dose amount, as used herein, is generally equal to the dosage of the active ingredient which may be administered once per day, or may be administered several times a day (e.g. the unit dose is a fraction of the desired daily dose). For example, a non-effective dose amount of 0.5 mg/day of S(−) pramipexole may be administered as 1 dose if 0.5 mg, 2 doses of 0.25 mg each or 4 doses of 0.125 mg. The term “unit dose” as used herein may be taken to indicate a discrete amount of the therapeutic composition which comprises a predetermined amount of the active compound. The amount of the active ingredient is generally equal to the dosage of the active ingredient which may be administered once per day, or may be administered several times a day (e.g. the unit dose is a fraction of the desired daily dose). The unit dose may also be taken to indicate the total daily dose, which may be administered once per day or may be administered as a convenient fraction of such a dose (e.g. the unit dose is the total daily dose which may be given in fractional increments, such as, for example, one-half or one-third the dosage).

A “No Observable Adverse Effect Level” (NOAEL) dose as used herein refers to an amount of active compound or pharmaceutical agent that produces no statistically or biologically significant increases in the frequency or severity of adverse effects between an exposed population and its appropriate control; some effects may be produced at this level, but they are not considered as adverse, or as precursors to adverse effects. The exposed population may be a system, animal, individual or human that is being treated by a researcher, veterinarian, medical doctor or other clinician. With respect to S(−) pramipexole, exemplary adverse events are dizziness, hallucination, nausea, hypotension, somnolence, constipation, headache, tremor, back pain, postural hypotension, hypertonia, depression, abdominal pain, anxiety, dyspepsia, flatulence, diarrhea, rash, ataxia, dry mouth, extrapyramidal snydrome, leg cramps, twitching, pharyngitis, sinusitis, sweating, rhinitis, urinary tract infection, vasodilation, flue syndrome, increased saliva, tooth disease, dyspnea, increased cough, gait abnormalities, urinary frequency, vomiting, allergic reaction, hypertension, pruritis, hypokinesia, nervousness, dream abnormalities, chest pain, neck pain, paresthesia, tachycardia, vertigo, voice alteration, conjunctivitis, paralysis, tinnitus, lacrimation, mydriasis and diplopia.

For example, a dose of 1.5 mg of S(−) pramipexole has been shown to cause somnolence in human subjects (Public Statement on Mirapex®, Sudden Onset of Sleep from the European Agency for the Evaluation of Medicinal Products; Boehringer Ingelheim product insert for Mirapex® which indicates that the drug is administered as three doses per day). Further, studies performed in dogs, as presented herein, (see Examples and results shown in Table 4) indicate that the NOAEL dose may be as low as 0.00125 mg/kg, which is equivalent to a human dose of 0.0007 mg/kg or 0.05 mg for a 70 kg individual. Thus, with reference to S(−) pramipexole, a NOAEL dose amount may be an amount below 1.5 mg, below 0.5 mg, or more preferably below 0.05 mg.

A “maximum tolerated dose” (MTD) as used herein refers to an amount of active compound or pharmaceutical agent which elicits significant toxicity in a tissue, system, animal, individual or human that is being treated by a researcher, veterinarian, medical doctor or other clinician. Single dose toxicity of S(−) pramipexole after oral administration has been studied in rodents, dogs, monkeys and human. In rodents, deaths occurred at doses of 70-105 mg/kg and above (Initial Scientific Discussion for the Approval of Mirapex from the European Agency for the Evaluation of Medicinal Products). This is equivalent to a human dose of 7-12 mg/kg, or approximately 500-850 mg for a 70 kg individual. Further, the Boehringer Ingelheim product insert for Mirapex® sets the maximally tolerated dose for humans at 4.5 mg/day. In human subjects, initial, single doses greater than 0.20 milligrams were not tolerated. In dogs, vomiting occurred at 0.0007 mg/kg and above while monkeys displayed major excitation at 3.5 mg/kg. All species showed signs of toxicity related to exaggerated pharmacodynamic responses to S(−) pramipexole. For example, behavioral changes including hyperactivity were common and led to a number of secondary effects, such as reduced body weight and other stress-induced symptoms. In minipigs an donkeys, S(−) pramipexole moderately affected cardiovascular parameters. In rats, the potent prolactin-inhibitory effect of pramipexole affected reproductive organs (e.g. enlarged corpora lutea, pyometra), and showed a dose-related retinal degeneration during long-term exposure (Initial Scientific Discussion for the Approval of Mirapex from the European Agency for the Evaluation of Medicinal Products).

Studies in dogs disclosed herein (see Examples and results in Table 4) indicate that the MTD may be as low as 0.0075 mg/kg, which is equivalent to a human dose of 0.0042 mg/kg or 0.30 mg for a 70 kg individual. Thus, with reference to S(−) pramipexole, a MTD amount for a human subject may be an amount below 4.5 mg/day, preferably below 1.5 mg/day. Further, the MTD amount for a human subject may be an amount below 0.3 mg/dose based on results of studies disclosed herein (see Table 4), and preferably below 0.2 mg/dose.

The terms “treating” may be taken to mean prophylaxis of a specific disorder, disease or condition, alleviation of the symptoms associated with a specific disorder, disease or condition and/or prevention of the symptoms associated with a specific disorder, disease or condition.

The term “patient” and “subject” are interchangeable and may be taken to mean any living organism which may be treated with compounds of the present invention. As such, the terms “patient” and “subject” may include, but is not limited to, any animal, mammal, primate or human.

Although any methods and materials similar or equivalent to those described herein can be used in the practice or testing of embodiments of the present invention, the preferred methods, devices, and materials are now described.

The compound 2-amino-4,5,6,7-tetrahydro-6-(propylamino)benzothiazole is a synthetic aminobenzothiazole derivative. The S(−) enantiomer, commonly known simply as pramipexole, is a potent dopamine agonist, with selective high affinity for the D₂, D₃ and D₄ subtypes of dopamine receptors. As a dopamine agonist, S(−) pramipexole activates dopamine receptors, thus mimicking the effects of the neurotransmitter dopamine. As such, S(−) pramipexole, which is commercially available as Mirapex®, is indicated for treating Parkinson's disease and restless legs syndrome.

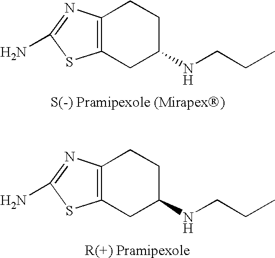

The S(−) pramipexole stereoismer is a potent agonist of dopamine, with only small daily doses required and tolerated by patients. The R(+) pramipexole stereoisomer, on the other hand, does not exhibit the same potent dopamine mimicking property, and may be tolerated in much higher doses. Both enantiomers, shown above, are able to confer neuroprotective effects by their ability to accumulate in brain cells, the spinal cord and mitochondria where they exert a positive effect on neurological function independent of the dopamine agonist activity, presumably through inhibition of lipid peroxidation, normalization of mitochondrial function and/or detoxification of oxygen radicals. As such, these compounds may have utility as inhibitors of the cell death cascades and loss of cell viability observed in neurodegenerative diseases. Clinical use of S(−) pramipexole as a mitochondria-targeted antioxidant is unlikely, however, since the high doses needed for this neuroprotective or anti-oxidative/mitochondrial normalization action are not achievable due to the side effects associated with excessive dopaminergic agonism. In contrast, S(−) pramipexole, which has been shown to be equally effective as S(−) pramipexole as a mitochondria-targeted neuroprotectant since both molecules show the same anti-oxidative properties, could be expected to be a clinically useful neuroprotectant due to its low affinity for dopamine receptors. The higher doses of the R(+) pramipexole that may be tolerated by patients without causing adverse side effects will allow greater brain, spinal cord and mitochondrial concentrations to be achieved and increase the degree to which oxidative stress and/or mitochondrial dysfunction may be reduced.

The high doses of R(+) pramipexole that may be required to achieve therapeutic efficacy will require very pure preparations of the R(+) enantiomer. Current clinical therapeutic doses of pramipexole (Mirapex®) are between 0.125 mg and 4.5 mg per day in order to reduce the frequency of its adverse side effects. As such, compositions of R(+) pramipexole for administration to subjects will need to be sufficiently chirally pure to take into account the upper limit of S(−) enantiomer tolerability in a given population.

Pramipexole appears to increase mitochondrial function in neural cells. For example, pramipexole has been shown to reduce the levels of free radicals produced by the parkinsonian neurotoxin and ETC complex I inhibitor methylpyridinium (MPP+) both in vitro and in vivo and has been reported to block opening of the mitochondrial transition pore (MTP) induced by MPP+ and other stimuli. Furthermore, both enantiomers of pramipexole restored calcein uptake in SH-SY5Y cells treated with MPP+.

In neural cells and an in vivo model of familial amyotrophic lateral sclerosis (ALS), pramipexole and its R(+) enantiomer have been shown to accumulate in mitochondria, to prevent mitochondrial injury, and to restore function.

R(+) pramipexole has anti-oxidant activity generally euipotent to that of pramipexole, but substantially lacks pharmacological dopaminergic activity. Therefore, R(+) pramipexole can be administered at higher dosages than S(−) pramipexole to achieve an antioxidative effect, while avoiding significant dopamine agonist activity.

R(+) pramipexole is a lipophilic cation that has been shown to cross cellular membranes and concentrate in mitochondria. Lipophilic cations pass easily through lipid bilayers because their charge is dispersed over a large surface area and the potential gradient drives their accumulation into the mitochondrial matrix. Fatty tissues and negatively charged cells provide ideal targets for this compound. R(+) pramipexole has anti-oxidant activity generally euipotent to that of S(−) pramipexole, but lacks the high dopamine receptor affinity and the corresponding pharmacological dopaminergic activity of its enantiomer. Therefore, R(+) pramipexole potentially can be administered at higher dosages than S(−) pramipexole to achieve an antioxidant effect, while avoiding clinically significant dopamine agonist activity.

Embodiments of the present invention relate to methods of using or administering R(+) pramipexole for the treatment and/or prevention of diseases and conditions associated with or involving decreased mitochondrial function or mitochondrial dysfunction. Such diseases and conditions include, but are not limited to, age-related macular degeneration, type II diabetes, skin diseases and disorders, coronary and cardiovascular diseases and disorders, and inflammatory disorders.

Further embodiments of the present invention relate to the use of R(+) pramipexole in the manufacture or preparation of a medicament for the treatment and/or prevention of diseases and conditions associated with or involving decreased mitochondrial function or mitochondrial dysfunction or increased oxidative stress. Such diseases and conditions include, but are not limited to, age-related macular degeneration, type II diabetes, skin diseases and disorders, coronary and cardiovascular diseases and disorders, and inflammatory disorders.

A preferred embodiment of the present invention relates to methods of using or administering R(+) pramipexole for the treatment and/or prevention of diseases and conditions associated with or involving decreased mitochondrial function or mitochondrial dysfunction. Such diseases and conditions include, but are not limited to, age-related macular degeneration, type II diabetes, skin diseases and disorders, coronary and cardiovascular diseases and disorders, and inflammatory disorders. In preferred embodiments, the methods include administering a pharmaceutical composition comprising R(+) pramipexole, more preferably a pharmaceutical composition with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%.

Further embodiments of the present invention relate to methods of using or administering R(+) pramipexole for the treatment and/or prevention of diseases and conditions associated with increased oxidative stress. Such diseases and conditions include, but are not limited to, age-related macular degeneration, type II diabetes, skin diseases and disorders, coronary and cardiovascular diseases and disorders, and inflammatory disorders.

Further preferred embodiments of the present invention relate to methods of using or administering R(+) pramipexole for the treatment and/or prevention of diseases and conditions associated with increased oxidative stress. Such diseases and conditions include, but are not limited to, age-related macular degeneration, type II diabetes, skin diseases and disorders, coronary and cardiovascular diseases and disorders, and inflammatory disorders. In preferred embodiments, the methods include administering a pharmaceutical composition comprising R(+) pramipexole, more preferably a pharmaceutical composition with a chiral purity for the R(+) enantiomer of greater than 80%, preferably greater than 90%, more preferably greater than 95%, and most preferably greater than 99%, including 99.5%, or greater, 99.6% or greater, 99.7% or greater, 99.8% or greater, 99.9% or greater, preferably 99.95% or greater and more preferably 99.99% or greater, or 100%.

Preferred embodiments of the present invention relate to compositions comprising pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater. In more preferred embodiments, the chiral purity for the R(+) enantiomer of pramipexole in the compositions may be 100%.

Embodiments of the present invention include compositions comprising R(+) pramipexole. In embodiments, the R(+) pramipexole may be a salt of R(+) pramipexole. In additional embodiments, the compositions may further comprise a pharmaceutically acceptable carrier.

Embodiments of the invention include compositions that may be administered orally, preferably as a solid oral dose, and more preferably as a solid oral dose that may be a capsule or tablet. In preferred embodiments, the compositions of the present invention may be formulated as tablets for oral administration.

Embodiments of the invention include pharmaceutical compositions comprising R(+) pramipexole and a no observable adverse effect level (NOAEL) dose amount of S(−) pramipexole. The pharmaceutical compositions of embodiments may be effective as inhibitors of oxidative stress, inhibitors of lipid peroxidation, in the detoxification of oxygen radicals and as neuroprotectants and other cellular protectants. In embodiments, the NOAEL dose amount of S(−) pramipexole may be an amount that does not exceed 1.50 mg. In additional embodiments, the NOAEL dose amount of S(−) pramipexole may be an amount that doe snot exceed 0.5 mg more preferably 0.05 mg.

Additional embodiments of the invention include pharmaceutical compositions comprising R(+) pramipexole and a non-effective dose amount of S(−) pramipexole. In embodiments, the non-effective dose amount of S(−) pramipexole may be an amount below 1.0 mg/day, below 0.75 mg/day, below 0.5 mg/day, below 0.25 mg/day, or preferably below 0.125 mg/day.

Further embodiments of the invention include pharmaceutical compositions comprising a therapeutically effective amount of R(+) pramipexole and a non-effective dose amount of S(−) pramipexole. In embodiments, the therapeutically effective amount of R(+) pramipexole may be from about 0.1 mg/kg/day to about 1,000 mg/kg/day or from about 1 mg/kg/day to about 100 mg/kg/day. In preferred embodiments, the therapeutically effective amount of R(+) pramipexole may be from about 3 mg/kg/day to about 70 mg/kg/day. In more preferred embodiments, the therapeutically effective amount of R(+) pramipexole may be from about 7 mg/kg/day to about 40 mg/kg/day. In other embodiments, the therapeutically effective amount of R(+) pramipexole may be from about 50 mg to about 5,000 mg, from about 100 mg to about 3,000 mg, preferably from about 300 mg to about 1,500 mg, and more preferably from about 500 mg to about 1,000 mg.

Additional embodiments of the invention include a pharmaceutical composition comprising a therapeutically effective amount of R(+) pramipexole and a NOAEL dose amount of S(−) pramipexole.

Yet additional embodiments of the invention include pharmaceutical compositions suitable for oral administration comprising a therapeutically effective amount of R(+) pramipexole and a non-effective dose amount of S(−) pramipexole. In embodiments, the pharmaceutical compositions suitable for oral administration comprise a therapeutically effective amount of R(+) pramipexole and a NOAEL dose amount of S(−) pramipexole.

In one embodiment, a method of treating or preventing macular degeneration or age-related macular degeneration comprising administering R(+) pramipexole is provided. The R(+) pramipexole may be administered in a composition, preferably a pharmaceutical composition, containing a therapeutically effective amount of R(+) pramipexole. More preferably, the method comprises administering a pharmaceutical composition comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, more preferably for ocular administration. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating age-related macular degeneration. The pharmaceutical composition may further comprise S(−) pramipexole in an amount that does not provide significant dopamine agonist activity. In another embodiment, the pharmaceutical composition consists essentially of R(+) pramipexole.

Age-related macular degeneration (AMD) is a degenerative condition of the macular, which is a cone-rich region of the central retina. Although the pathogenesis of the disease is unknown, numerous studies have suggested that oxidative stress plays a prominent role in the disease. Oxidative stress is defined as cellular injury associated with reactive oxygen species (ROS).

The retina has been described as an ideal environment for the generation of ROS because of: (1) its exposure to cumulative radiation; (2) the high concentration of polyunsaturated fats in the outer segment membrane; (3) the abundance of photosensitizers in the retinal pigment epithelium (RPE); and (4) its increased oxygen consumption compared to other tissues. In addition, phagocytosis by the RPE not only promotes oxidative stress directly, but also creates additional ROS, which can cause further injury.

Both the production of ROS and the stress associated with their production is concentrated in the mitochondria. Mitochondrial DNA (mtDNA) is particularly susceptible to oxidative modification, possesses inferior repair systems, and exists in close proximity to the site of ROS-generation. Mitochondrial damage as a result of oxidative stress can result in reduced cellular energy production, comprised cell function, and apoptosis. Most risk factors associated with AMD share oxidative stress as a common denominator. These include low nutritional consumption of anitoxidants, exposure to cigarette smoke, and exposure to sunlight.

In healthy subjects, the stress associated with the concentration of mitochondrial ROS in the retina and macular is mitigated by high concentrations of antioxidant agents, particularly in the RPE layer. These include vitamin E, superoxide dismutase, catalase, glutathione-S-transferases, glutathione, ascorbate, and zinc. However, the ability of RPE cells to mount a defense to natural oxidative processes appears to diminish with age.

Without wishing to be bound by theory, it is believed that the protective and restorative effects of the compositions described herein derive at least in part from R(+) pramipexole's ability to prevent retinal cell death by at least one of three mechanisms: (1) the R(+) enantiomer is capable of reducing the formation of reactive oxygen species (ROS) or functioning as free radical scavengers; (2) the R(+) enantiomer can partially restore the reduced mitochondrial activity associated with oxidative stress in the retina, the macular, or the RPE layer; and (3) the R(+) enantiomer can block the apoptotic cell death pathways produced in models of AMD. The R(+) enantiomer of pramipexole is a lipophilic cation that has been shown to cross neuronal membranes and concentrate in neuronal mitochondria. The high lipid concentration of the retina, macular, and particularly the RPE, and the negative charge of retinal cells provide an ideal target for the compound.

In another embodiment, a method of treating or preventing type II diabetes comprising administering R(+) pramipexole is provided. The R(+) pramipexole may be administered in a composition, preferably a pharmaceutical composition, containing a therapeutically effective amount of R(+) pramipexole. More preferably, the method comprises administering a pharmaceutical composition comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, such as a capsule or tablet. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating type II diabetes. The pharmaceutical composition may further comprise S(−) pramipexole in am amount that does not provide significant dopamine agonist activity. In another embodiment, the pharmaceutical composition consists essentially of R(+) pramipexole.

In a further embodiment, a method of treating or preventing insulin resistance comprising administering R(+) pramipexole is provided. The R(+) pramipexole may be administered in a composition, preferably a pharmaceutical composition, containing a therapeutically effective amount of R(+) pramipexole. More preferably, the method comprises administering a pharmaceutical composition comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, such as a tablet or capsule. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating insulin resistance. The pharmaceutical composition may further comprise S(−) pramipexole in an amount that does not provide significant dopamine agonist activity. In another embodiment, the pharmaceutical composition consists essentially of R(+) pramipexole.

Type II diabetes and insulin resistance are both involved in various diseases, disorders and conditions, which therefore may be treated, controlled or prevented with the compositions of the present invention, including, hyperglycemia, low glucose tolerance, obesity, lipid disorders, dyslipidemia, coronary heart disease, hyperlipidemia, hypertriglyceridemia, hypercholesterolemia, hypertension, low HDL levels, high LDL levels, atherosclerosis and its sequelae, vascular stenosis and restenosis, irritable bowel syndrome, inflammatory bowel disease, including Crohn's disease and ulcerative colitis, other inflammatory conditions, pancreatitis, abdominal obesity, neurodegenerative disease, retinopathy, nephropathy, neuropathy, Syndrome X (metabolic syndrome), ovarian hyperandrogenism (polycystic ovarian syndrome), and other disorders where insulin resistance is a component. In Snydrome X, obesity is thought to promote insulin resistance, diabetes, dyslipidemia, hypertension, and increased cardiovascular risk.

Type II diabetes is a disease process derived from multiple causative factors and characterized by elevated levels of plasma glucose or hyperglycemia in the fasting state or after administration of glucose during an oral glucose tolerance test. Persistent or uncontrolled hyperglycemia is associated with increased and premature morbidity and mortality. Often abnormal glucose homeostatis is associated both directly and indirectly with alterations of lipid, lipoprotein and apoliproproten metabolism and other metabolism and hemodynamic disease. Therefore patient with type II diabetes mellitus are at increased risk of developing various other conditions, including coronary heart disease, stroke, peripheral vascular disease, hypertension, nephropathy, neuropathy, and retinopathy.

Insulin resistance is known to be an antecedent condition to type II diabetes. There is accumulating scientific evidence that impaired mitochondrial activity may be a factor in insulin resistance. Specifically, evidence supports the existence of an inherited genetic dysfunction in intramyocellular fatty acid metabolism in offspring of patients with type II diabetes. The defect appears to be linked to defects in mitochondrial phosphorylation, which may be due to reduced mitochondrial content.

In another embodiment, a method of treating or preventing skin conditions or disorders comprising administering R(+) pramipexole is provided. The R(+) pramipexole may be administered in a composition, preferably a pharmaceutical composition or a cosmetic preparation, containing a therapeutically effective amount of R(+) pramipexole. More preferably, the method comprises administering a pharmaceutical composition or cosmetic preparation comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.5% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, more preferably for topical administration. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating skin disorders or conditions. The pharmaceutical composition may further comprise S(−) pramipexole in an amount that does not provide significant dopamine agonist activity. In another embodiment, the pharmaceutical composition consists essentially of R(+) pramipexole.

A further embodiment provided is a method of enhancing or improving the appearance of skin, such as by reduction or removal of facial lines, wrinkles and stretch marks by administering R(+) pramipexole. The R(+) pramipexole may be administered in a composition, preferably a pharmaceutical composition or cosmetic preparaiton, containing a therapeutically effective amount of R(+) pramipexole. More preferably, the method comprises administering a pharmaceutical composition or cosmetic preparation comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, ore preferably for topical administration. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in enhancing or improving the appearance of skin, such as by reduction or removal of facial lines, wrinkles and stretch marks. The pharmaceutical composition may further comprise S(−) pramipexole in an amount that does not provide significant dopamine agonist activity. In another embodiment, the pharmaceutical composition consists essentially of R(+) pramipexole.

The skin, continuously exposed to sunlight and environmental oxidizing pollutants, is a primary site of oxidative stress in humans. Substantial evidence links cumulative oxidative stress to familiar signs of skin aging, including wrinkling, sagging, hyperplasia, and actinic lentigo, as well as to such medical pathologies as melanoma, psoriasis, and scleroderma. It is widely accepted that ultraviolet irradiation and environmental chemical and physical agents induce the formation of ROS in cutaneous tissues, provoking lipid peroxidation, protein cross-linking, enzyme inactivation, apoptosis, and other pathological effects. Thinning of the atmospheric ozone layer has resulted in increased exposure of irradiation at wavelengths demonstrated to penetrate the epidermis. Apart from such exogenous factors, the epidermis itself is a major producer of oxidative molecules through metabolism,

In skin, as in other organs, both the production of ROS and the stress associated with their production is concentrated in the mitochondria. The primary function of the mitochondria is the generation of ATP through oxidative phosphorylation via the electron transport chain. mtDNA is particularly susceptible to oxidative modification, which can result in reduced cellular energy production, compromised cell function, and apoptosis. ROS generated by UV irradiation can also damage nuclear DNA, causing mutations in growth regulatory genes that lead to the loss of cell-cycle control, DNA repair, and regulation of apoptosis. In addition, ROS action has been demonstrated to interfere with immune response to cutaneous tumors.

To counteract oxidative injury, skin cells are equipped with a network of enzymatic and non-enzymatic antioxidant systems. However, endogenous antioxidant systems in the mitochondria have been shown to diminish with age through telomere shortening, carbonyl aconitase modificaiton, cumulative UV irradiation, and other mechanisms. Thus, both chronological aging and photaging play a role in the promotion of oxidative stress in the mitochondria of skin cells and in the dysfunction of anti-oxidant mechanisms.

Without wishing to be bound by theory, the protective and restorative effects of the embodiments of the present invention may derive at least in part from R(+) pramipexole's ability to prevent the effects of aging or pathology in skin cells by at least one of three mechanisms. First, R(+) pramipexole may reduce the formation of ROS or functioning as free radical scavengers. Second, R(+) pramipexole may partially restore the reduced mitochondrial activity associated with oxidative stress in cutaneous tissue. Third, R(+) pramipexole may lock the apoptotic cell death pathways produced in models of aging and skin disease, including melanoma and other neoplasias.

In another preferred embodiment, a method of treating or preventing coronary or cardiovascular diseases comprising administering R(+) pramipexole is provided. The R(+) pramipexole may be administered in a composition, preferably a pharmaceutical composition, containing a therapeutically effective amount of R(+) pramipexole. More preferably, the method comprises administering a pharmaceutical composition comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.5% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, such as a tablet or capsule. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating coronary or cardiovascular diseases. Such coronary or cardiovascular diseases include, but are not limited to, myocardial infarction, congestive heart failure, atherosclerosis, hypertension, adverse effects of CABG therapy, coronary heart disease, vascular restenosis, acute myocardial infarction, and ischemic repercussion injury. The pharmaceutical composition may further comprise S(−) pramipexole in an amount that does not provide significant dopamine agonist activity. In another embodiment, the pharmaceutical composition consists essentially of R(+) pramipexole.

Heart failure and associated conditions, including vascular dementia and other diseases of the cardiovascular system, are associated with oxidative stress in the mitochondria. Mitochondria produce damaging ROS as a consequence of electrons leaking in the electron transport chain. mtDNA in the heart, as in other tissues, is vulnerable to oxidative stress because of its proximity to ROS production and the absence of histones that protect nuclear DNA. ROS-induced mutations of mtDNA affect electron transport, which not only reduces the capacity to synthesize ATP but increases further ROS production. Damage to proteins, including antioxidant enzymes, has also been observed to promote mitochondrial dysfunction. Moreover, post-mitotic cells such as cardiac myocytes create an environment that promotes increasing accumulation of mtDNA deletions and mutations. In blood vessels ROS induce both contraction and endothelial dysfunction and cause hypertrophic remodeling.

The heart is particularly vulnerable to mitochondrial dysfunction because of myocardial dependency on oxidation for energy. The heart maintains low reserves of ATP, making the continuous production of ATP essential for myocardial function. Both systolic contraction and diastolic relaxation require high levels of ATP. Reductions in ATP compromise CA2+ reuptake from the cytosol among other ways of compromising normal cardiac mechanics.

The destructive effects of myocardial oxidative stress include disruption and collapse of the inner mitochondrial membrane potential, which promotes apoptosis, as well as hypertrophic remodeling of the myocardium. A reduction in membrane potential has been observed to increase with age. Increased production of superoxide and hydrogen peroxide has been observed in the myocytes of old rats. Diminished mitochondrial turnover in older subjects depresses phagocytic capacity, which in turn promotes increased production of ROS. Theories of oxidative stress and its effect on myocardial dysfunction are supported by studies in which antioxidant compounds, including synthesized compounds and natural compounds abundant in fruits, are correlated with reduced incidence of cardiac and cardiovascular disease.

Some therapeutic approaches to cardiovascular disease actually result in acute oxidative stress. These therapies include coronary artery bypass grafting (CABG), during which an elevated incidence of biomarkers of oxidative stress is observed during and immediately following CABG therapy. Some investigators have accordingly called for the development of early counter-regulators of free radical reactions during CABG or other procedures that introduce the risk of ischemic repercussion injury. The pathological effects of oxidative stress are present in numerous additional diseases of the cardiovascular system. These include, for example, atherosclerosis, congestive heart failure, and hypertension.

The vascular endothelium plays a central role in the regulation of vascular function. In particular, the local release of endothelium-derived relaxing factor (EDRF) regulates vascular tone and prevents platelet adhesion to the vascular wall. Impairment of EDRF action develops early in atherosclerosis and, in part, contributes to platelet deposition and vasospasm involved in the clinical expression of coronary artery disease. Recent evidence suggests that an imbalance between vascular oxidative stress and antioxidant protection is involved in the development of this vascular dysfunction. ROS are generated by enzyme systems presnet in cells in the vascular wall, including NADPH oxidase, xanthine oxidase, and nitric oxide synthase. The activities and levels of these enzyme systems are increased in association with vascular disease risk factors.

Research demonstrates a progressive increase in free radical injury and encroachment on antioxidant reserves with the evolution of heart failure. Oxidative stress has been identified as an important determinant of prognosis. In animal models, the development of congestive heart failure (CHF) is accompanied by changes in the antioxidant defense mechanisms of the myocardium as well as evidence of oxidative myocardial injury.

Elevated ROS has been observed in hypertension, frequently with impairment of endogenous antioxidant mechanisms. Experimental manipulation of the redox state in vivo shows that ROS can cause hypertension. During the development of hypertension, ROS are generated by endogenous sources, notably NADPH oxidase enzymes and uncoupled nitric oxide synthase, due to a mutual reinforcement between ROS and humoral factors. ROS also promote renal salt reabsorption and decrease glomerular filtration.

Without wishing to be bound by theory, the protective and restorative effects of the may derive at least in part from the active compound's ability to address cardiac or cardiovascular disease by at least one of three mechanisms. First, R(+) pramipexole may reduce the formation of ROS or function as a free radical scavenger. Second, R(+) pramipexole may partially restore the reduced mitochondrial activity associated with oxidative stress in cardiomyocytes, in the vascular epithelium, and other cardiovascular tissues. Third, R(+) pramipexole may block apoptotic cell death pathways produced in heart and cardiovascular disease.

In another embodiment, a method of treating or preventing inflammatory disorders comprising administering R(+) pramipexole is provided. The R(+) pramipexole may be administered in a composition, preferably a pharmaceutical composition, containing a therapeutically effective amount of R(+) pramipexole. More preferably, the method comprises administering a pharmaceutical composition comprising a therapeutically effective amount of R(+) pramipexole with a chiral purity for the R(+) enantiomer of greater than 80%, preferably greater than 90%, more preferably greater than 95%, and most preferably greater than 99%, including 99.5% or greater, 99.6% or greater, 99.7% or greater, 99.8% or greater, 99.9% or greater, preferably 99.95% or greater and more preferably 99.99% or greater, or 100%. The therapeutically effective amount of R(+) pramipexole may be from about 50 milligrams to about 5000 milligrams, about 100 milligrams to about 3000 milligrams, preferably from about 300 milligrams to about 1500 milligrams, more preferably from about 500 milligrams to about 1000 milligrams. The pharmaceutical composition may be suitable for oral administration, such as a tablet or capsule. In other embodiments, the pharmaceutical composition may contain a no observable adverse effect level amount of S(−) pramipexole or a non-effective dose amount of S(−) pramipexole. In a further embodiment, the pharmaceutical composition may further comprise an agent useful in treating inflammatory related disorders. Inflammatory related disorders resulting from oxidative stress include but are not limited to trauma, trauma due to surgery, burns, acute respiratory distress syndrome, pancreatitis, sepsis and Systemic Inflammatory Response Syndrome (SIRS). The pharmaceutical composition may further comprise S(−) pramipexole in an amount that does not provide significant dopamine agonist activity. In another embodiment, the pharmaceutical composition consists essentially of R(+) pramipexole.

Dysfunction of the inflammatory response may turn a protective mechanism into a deadly one. Generally, inflammation is localized to the area of injury or infection. However, in some instances production of pro-inflammatory factors may be accelerated and the area of inflammation may be extended outside of the area of injury. Systemic Inflammatory Response Syndrome (SIRS) describes a disorder in which an inflammatory response is activated systemically, causing runaway inflammation throughout the body and eventually resulting in multi-organ failure, loss of vascular patency, and shock. SIRS encompasses a family of diseases with multiple etiologies being initiated, for example, by trauma, surgery, burns, acute respiratory distress syndrome, and pancreatitis. The most prevalent manifestation of SIRS involves infectious etiology, a condition called sepsis.

The production of excess ROS has been identified as an initiating, enhancing, and damaging factor in sepsis and other SIRS-related diseases. Elevated ROS production in sepsis has been associated with dysfunction mitochondrial respiratory electron transport chain, excess production of xanthine oxidase as a result of ischemic/repercussion activity, activaiton of immune cells and associated respiratory activity, and metabolism of arachadonic acid.

ROS act as molecular triggers of systemic inflammation by promoting the generation of cytokines. ROS also prepare endothelial cells to recruit inflammatory cells and also cause tissue damage, which further promotes inflammatory response. At the initiatory stage, cellular oxidative stress plays a key role in the generation of pro-inflammatory cytokines. Agents of cyctokine production include NF-κB, a transcription factor involved in the regulation of pro-inflammatory genes. TNF-α and IL-6, two of the most prominent pro-inflammatory cytokines, have been shown to be regulated by NF-κB activation, particularly in sever pancreatitis. In several in vitro and in vivo models, a link has been established between NF-κB activation and sepsis. Indeed, NF-κB levels and accompanying increases in cytokine activity have been shown to correspond with APACHE II scores, the best available predictor of outcome and mortality from sepsis.

ROS activate other transcription factors that in turn regulate inflammatory genes. ROS induce phosphorylation of mitogen activated protein kinases (MAP kinases), including ERK, JNK, and p30 kinases. MAP kinases are also believed to regulate histone acetylation and phosphorylation, which play a role in the production of the pro-inflammatory cytokines IL-2 and IL-8.

In addition to ROS, reactive nitrogen species (RNS) act as activators and promoters of systemic inflammation. Nitric oxide produced by activated macrophages represents an essential protective component of the inflammatory process. However, NO and other RNS promote tissue injury which further promotes the inflammatory response. NO also stimulates the production of hydrogen peroxide and oxygen free radicals in mitochondria through leakage of electrons from the transport chain. In a vicious cycle, hydrogen peroxide, in turn, promotes iNOS expression through NK-κB activation.

In addition to their role in initiating inflammation, ROS promote the spread of inflammation to non-local or non-specific injury sites. Local insults, such as surgery, generate the production of neutrophils, which may travel to and become sequestered in distal organs. The systemic activity of neutrophils also promotes inflammation in large areas of endothelium, where bound neutrophils release proteases and additional ROS. The ROS generated by neutrophils promote secondary injury incident to surgery and other interventions. The effects of endothelial inflammation include the initiation of a secondary inflammatory cascade and the stimulation of further cytokine production.

The presence of neutrophils in distal organs destroys the homeostatic balance between proteases and anti-proteases, which reduces cellular viability and promotes degradation of the extracellular matrix, both of which are associated with organ failure. Certain additional cytokines promote oxidative stress and contribute to the injury of distal organs. In serious burn patients, for example, so-called cytokine “storms” are associated with secondary cardiac injury.

The dysfunction of the anti-inflammatory response is complex, but may involve down-regulation of agents that mediate ROD and RNS, particularly in the mitochondria. For example, sepsis patients exhibit reduced concentrations of endogenous anitoxidants, including vitamin A and vitamin E. As a result, anitoxidants that concentrate within pro-inflammatory cells and within the mitochondria of organ cells have been described as compelling therapeutic candidates for the treatment of complications associated with systemic inflammatory response.

Without wishing to be bound by theory, the preventive and protective effects associated with the compositions of the invention may be derived at least in part from the ability of R(+) pramipexole to regulate inflammatory response through inhibition of pro-inflammatory mediators, such as, for example, neutrophils, macrophages, cytokines, and the like, as well as transcription factors associated with these mediators, including but not limited to NF-κB. The compositions of the invention may also reduce the formation of ROS and RNS or act as a free radical scavenger, thereby attenuating the inflammatory response in response to local insult, and may inhibit the initiation, spread, and acceleration of systemic inflammatory response by regulating the activity of neutrophils in endothelial tissue and the systemic activity of cytokines. Therefore, the compositions of the invention may be capable of preventing secondary effects of local and systemic inflammatory response and protecting distal organs. Moreover, R(+) pramipexole, as a lipophilic cation, may be capable of penetrating cellular membranes and concentrating in mitochondria, taking it to sites of cytokine activation.

Each of the foregoing preferred embodiments may employ the use of compositions comprising pramipexole which is chirally pure for the R(+) enantiomer, or a pharmaceutically acceptable salt thereof. The compositions may be administered to subjects in doses that range from between 0.1 mg/kg/day to 1,000 mg/kg/day. Preferably, the compositions may be administered in doses of from about 50 mg to about 5,000 mg, from about 100 mg to about 3,000 mg, from about 300 mg to about 1,500 mg, or from about 500 mg to about 1,000 mg. These doses of pramipexole preferably are in preparations which have a chemical purity of greater than 80%, preferably greater than 90%, more preferably greater than 95%, greater than 97%, and most preferably greater than 99%, including 99.5% or greater, 99.6% or greater, 99.7% or greater, 99.8% or greater, 99.9% or greater, preferably 99.95% or greater and more preferably 99.99% or greater. IN a preferred embodiment, the compositions comprising pramipexole, or a pharmaceutically acceptable salt thereof, may have a chiral purity for the R(+) enantiomer of 100%. The compositions may further comprise a carrier. The compositions of the present invention may be administered orally, preferably as a solid oral dose, and more preferably as a solid oral dose that may be a capsule or tablet. In preferred embodiments, the compositions of the present invention may be formulated as tablets for oral administration.

The need for pramipexole compositions of such high chiral purity for the R(+) enantiomer is apparent from the experimental data disclosed herein (see Examples and Tables 3 and 4). Previous data in the literature indicate that the R(+) enantiomer of pramipexole is 10 to 200-fold less active as a dopamine receptor agonist than the S(−) enantiomer. Unexpectedly this reported ratio may greatly underestimate the different affinities of the R(+) and S(−) enantiomers of pramipexole for the dopamine receptors (see Examples), and thereby fails to appreciate the degree of chiral purity necessary to make R(+) pramipexole practical or suitable as a therapeutic composition. In fact, as shown in Table 3, the R(+) enantiomer may be from more than 5,000-fold to greater than 10,000 fold less active as a dopamine agonist than the S(−) enantiomer of pramipexole (Table 3). Furthermore, in animal studies, the NOAEL dose for the R(+) enantiomer is 20,000-fold greater than for the S(−) enantiomer (Table 4). Thus, for compositions of pramipexole which are chirally pure for the R(+) enantiomer, even a small (fractional percentage) contamination with the S(−) enantiomer may have observable and predictable adverse consequences.

While not wishing to be bound by theory, these data (see Examples and Tables 3 and 4) present a number of interesting possibilities. Initially, the data demonstrate the high (approaching absolute) chiral purity of the pramipexole compositions for the R(+) enantiomer. R(+) pramipexole is administered in high dose levels in the studies disclosed herein (equivalent to human doses of 1,000 mg to 3,000 mg; see Examples), so that even the smallest amount of S(−) pramipexole would contribute to the observed NOAEL and MTD. For example, with reference to human equivalence doses based on data obtained in dogs, the MTD for the R(+) enantiomer has been shown to be equivalent to about 3,000 mg for a 70 kg human subject, while the equivalent MTD for the S(−) enantiomer would be equivalent to only 0.30 mg for that same subject (Table 4). That is a difference of 10,00-fold. As mentioned above, the NOAEL dose for the R(+) enantiomer is 20,000-fold greater than for the S(−) enantiomer (Table 4). Thus the R(+) pramipexole compositions used in these studies must be at least 99.99% pure if one were to assume that the observed side effects stemmed only from contamination by the S(−) enantiomer. On the other hand, these data demonstrate the high dose levels of the R(+) enantiomer of pramipexole that may be administered safely. These data highlights the importance of the high chiral purity for the R(+) enantiomer of pramipexole that may be used in various aspects of the present invention.

The R(+) pramipexole of the present invention may be synthesized and/or purified by methods disclosed in the copending U.S. Provisional Application No. 60/894,829 entitled “Methods of Snythesizing and Purifying R(+) and S(−) pramipexole”, filed Mar. 14, 2007, and U.S. Provisional Application No. 60/894,814 entitled “Methods of Enantiomerically Purifying Chiral Compounds”, filed Mar. 14, 2007, which are incorporated herein by reference in their entireties. Specifically, preparations of pramipexole which are chirally pure for the R(+) enantiomer may be produced using a bi-molecular nucleophilic substitution (S_{N}2) reaction. The process comprises dissolving a diamine of formula 2,6 diamino-4,5,6,7-tetrahydro-benothiazole in an organic solvent, reacting the diamine with a propyl sulfonate or a propyl halide under conditions sufficient to generate and precipitate the pramipexole salt, and recovering the pramipexole salt. In an embodiment, the propyl sulfonate may be propyl tosylate. The conditions sufficient to generate and precipitate the pramipexole salt comprise using dimethylformamide as the organic solvent and heating the dissolved diamine at an elevated temperature. A mixture of propyl sulfonate or propyl halide, preferably about 1.25 molar equivalents, dissolved in dimethylformamide, preferably at about 10 volumes, and di-isoproplyethylamine, preferably at about 1.25 molar equivalents, is added slowly to the heated diamine with stirring over a period of several hours. Alternatively, the di-isoproplyethylamine may be added to the reaction with the diamine, and the propyl sulfonate or propyl halide may be dissolved in dimethylformamide to form a mixture, which may be added to the reaction with stirring for several hours. The elevated temperature of the reaction may be about 65° C. or lower. The times necessary for the reaction may vary with the identities of the reactants, the solvent system and with the chosen temperature, and may be understood by one skilled in the art.

Embodiments of the process further comprise cooling the reaction to about room temperature and stirring the reaction for several hours. The process may further involve filtering the reaction to isolate a solid precipitate, washing the precipitate with an alcohol, and drying the precipitate under vacuum. The pramipexole salt reaction product of this process displays a high chemical purity and an increased optical purity over the reactants. Without wishing to be bound by theory, the increased optical purity may be due to limited solubility of the pramipexole salt reaction product in the polar solvents of the reaction mixture. Purification of the final pramipexole reaction product from the reaction mixture thus involves simple trituration and washing of the precipitated pramipexole salt in a volatile solvent such as an alcohol or heptane, followed by vacuum drying.

The chemical and chiral purity of the preparations of R(+) pramipexole may be verified with at least HPLC, ¹³C-NMR, ¹H-NMR and FTIR. In preferred embodiments, the R(+) pramipexole may be synthesized by the method described above, which yields enantiomerically pure material. Alternatively, the R(+) pramipexole may be purified from mixtures of R(+) and S(−) pramipexole using a purification scheme which is disclosed in U.S. Provisional Application No. 60/894,829 entitled “Methods of Snythesizing and Purifying R(+) and S(−) pramipexole”, filed Mar. 14, 2007, and U.S. Provisional Application No. 60/894,814 entitled “Methods of Enantiomerically Purifying Chiral Compounds”, filed Mar. 14, 2007, which are incorporated herein by reference in their entireties. Pramipexole, which is chirally pure for the R(+) enantiomer, may be triturated from an enantiomerically enriched pramipexole acid addition solution based on insolubility of the enantiomeric salts in the resulting achiral reagents. Embodiments of the process comprise dissolving pramipexole which is enantiomerically enriched for the R(+) enantiomer in an organic solvent at an elevated temperature, adding from about 1.0 molar equivalents to about 2.0 molar equivalents of a selected acid, cooling the reaction to room temperature, stirring the cooled reaction at room temperature for an extended time and recovering enantiomerically pure R(+).

The chirally pure R(+) pramipexole prepared by either of the above methods may be converted to a pharmaceutically acceptable salt of R(+) pramipexole. For example, R(+) pramipexole dihydrochoride is a preferred pharmaceutical sale due its high water solubility. R(+) pramipexole dihydrochoride may be prepared from other salts of R(+) pramipexole in a one step method comprising reacting the R(+) pramipexole, or R(+) pramipexole salt, with concentrated HCl in an organic solvent, such as an alcohol, at a reduced temperature. A preferred reduced temperature is a temperature of from about 0° C. to about 5° C. An organic solvent, such as methyl tert-butyl ether, may be added, and the reaction may be stirred for an additional hour. The R(+) pramipexole dihydrochoride product may be recovered from the reaction mixture by filtering, washing with an alcohol and vacuum drying.

Each of the methods disclosed herein for the manufacture and purification of R(+) pramipexole or a pharmaceutically acceptable salt thereof may be scalable to provide industrial scale quantities and yields, supplying products with both high chemical and chiral purity. As such, in preferred embodiments, enantiomerically pure R(+) pramipexole may be manufactured in large batch quantities as may be required to meet the needs of a large scale pharmaceutical use.

The high chiral purity of the R(+) pramipexole used herein allows for therapeutic compositions that may have a wide individual and daily dose range. In one embodiment, the compositions of R(+) pramipexole may be used to treat R(+) neurodegenerative diseases, or other diseases associated with mitochondrial dysfunction or increased oxidative stress. The compositions of the present invention may also be useful in the treatment of other disorders not listed herein, and any listing provided in this invention is for exemplary purposes only and is non-limiting.

Compositions which comprise R(+) pramipexole may be effective as inhibitors of oxidative stress, inhibitors of lipid peroxidation, in the detoxification of oxygen radicals, and the normalization of mitochondrial function. Oxidative stress may be caused by an increase in oxygen and other free radicals.

Thus, the neuroprotective effect of the compositions of the present invention may drive at least in part from the ability of the R(+) enantiomer of pramipexole to prevent neural cell death by at least one of three mechanisms. First, the R(+) enantiomer of pramipexole may be capable of reducing the formation of reactive oxygen species in cells with impaired mitochondrial energy production. Second, the R(+) enantiomer of pramipexole may partially restore the reduced mitochondrial membrane potential that has been correlated with Alzheimer's disease, Parkinson's disease and amyotrophic lateral sclerosis diseases. Third, the R(+) enantiomer of pramipexole may block the cell death pathways which are produced by pharmacological models of Alzheimer's disease, Parkinson's disease, amyotrophic lateral sclerosis diseases and mitochondrial impairment.

As such, an embodiment of the invention is a composition comprising R(+) pramipexole, or a pharmaceutically acceptable salt thereof. The composition may further comprise a pharmaceutically acceptable carrier. An additional embodiment of the invention is a composition comprising a therapeutically effective amount of R(+) pramipexole, or a pharmaceutically acceptable salt thereof. The composition may further comprise a pharmaceutically acceptable carrier. An additional embodiment of the invention is a composition comprising a therapeutically effective amount of R(+) pramipexole, or a pharmaceutically acceptable salt thereof, and a non-effective dose amount of S(−) pramipexole. The therapeutic composition may further comprise a pharmaceutically acceptable carrier. An additional embodiment of the invention is a composition comprising a therapeutically effective amount of R(+) pramipexole, or a pharmaceutically acceptable salt thereof, and a no observable adverse effect level (NOAEL) amount of S(−) pramipexole. The therapeutic composition may further comprise a pharmaceutically acceptable carrier. The compositions of the invention may be administered orally, preferably as a solid oral dose, and more preferably as a solid oral dose that may be a capsule or tablet. In preferred embodiments, the compositions of the present invention may be formulated as tablets for oral administration.

An additional embodiment of the invention is a composition useful as a neuroprotectant comprising a therapeutically effective amount of R(+) pramipexole, or a pharmaceutically acceptable salt thereof. The composition may further comprise a pharmaceutically acceptable carrier. The composition may be useful in the treatment of diseases which may be alleviated by the action of a neuroprotectant.

Further compositions of the present invention are also described in U.S. Provisional Application No. 60/894,799 entitled “Modified Release Formulations and Methods of Use of R(+) Pramipexole” filed Mar. 14, 2007, herein incorporated by reference in its entirety. Specifically, the compositions comprising R(+) pramipexole may be formulated into modified release formulations, which are capable of releasing a therapeutically effective amount of R(+) pramipexole over an extended period of time, preferably at least about eight hours, more preferably at least about twelve hours, and even more preferably about twenty-four hours. Delayed release, extended release, controlled release, sustained release and pulsatile release dosage forms and their combinations are types of modified release dosage forms.

The compositions of these several embodiments which comprise R(+) pramipexole as an active agent may be effective as inhibitors of oxidative stress, inhibitors of lipid peroxidation, in the detoxification of oxygen radicals, and the normalization of mitochondrial function. Further, they may be effective as treatment for impaired motor function, and in degenerative diseases that may affect cardiac and striated muscle and retinal tissues.

Yet another embodiment of the invention is a method for treating a neurodegenerative disease by administering a therapeutically effective amount of R(+) pramipexole. In accordance with this embodiment, the R(+) pramipexole may be formulated as a pharmaceutical or therapeutic composition by combining with one or more pharmaceutically acceptable carriers. Embodiments include pharmaceutical or therapeutic compositions that may be administered orally, preferably as a solid oral dose, and more preferably as a solid oral dose that may be a capsule or tablet. In a preferred embodiment, the pharmaceutical or therapeutic composition is formulated in tablet or capsule form for use in oral administration routes. The compositions and amounts of non-active ingredients in such a formulation may depend on the amount of the active ingredient, and on the size and shape of the table or capsule. Such parameters may be readily appreciated and understood by one of skill in the art.

The pharmaceutical or therapeutic compositions may be prepared, packaged, sold in bulk, as a single unit dose, or as multiple unit doses.

For the purposes of this invention, a “salt” of the R(+) pramipexole, as used herein is any acid addition salt, preferably a pharmaceutically acceptable acid addition salt, including but not limited to, halogenic acid salts such as, for example, hydrobromic, hydrochloric, hydrofluoric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesulfonic), acetic, malic, fumaric, succinic, citric, benzoic, gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogenic, di-sulfuric, di-phosphoric or di-organic acid salt. In all cases, the acid addition salt is used as an achiral reagent which is not selected on the basis of any expected or known preference for interaction with or precipitation of a specific optical isomer of the products of this invention (e.g. as opposed to the specific use of D(+) tartaric acid in the prior art, which may preferentially precipitate the R(+) enantiomer of pramipexole).

“Pharmaceutically acceptable salt” is meant to indicate those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. (1997) J. Pharm. Sciences, Vol 6. 1-19, described pharmaceutically acceptable salts in detail.

The compositions may be formulated to be administered orally, ophthalmically, intravenously, intramuscularly, intra-arterially, intramedularry, intrathecally, intraventricularly, transdermally, subcutaneously, intraperitoneally, intravesicularly, intranasally, enterally, topically, sublingually, or rectally. In embodiments, the therapeutically effective amount of R(+) pramipexole may be from about 0.1 mg/kg/day to about 1,000 mg/kg/day or from about 1 mg/kg/day to about 100 mg/kg/day. In preferred embodiments, the therapeutically effective amount of R(+) pramipexole may be from about 3 mg/kg/day to about 70 mg/kg/day. In more preferred embodiments, the therapeutically effective amount of R(+) pramipexole may be from about 7 mg/kg/day to about 40 mg/kg/day. In embodiments, the therapeutically effective amount of R(+) pramipexole may be from about 50 mg to about 5,000 mg, from about 100 mg to about 3,000 mg, preferably from about 300 mg to about 1,500 mg, or more preferably from about 500 mg to about 1,000 mg.

In embodiments, the non-effective dose amount of S(−) pramipexole is an amount that does not exceed a total dose of 1.0 mg/day. In more preferred embodiments, the non-effective dose amount of S(−) pramipexole is an amount that does not exceed a total dose of 0.75 mg/day, 0.5 mg/day, 0.25 mg/day, and preferably 0.125 mg/day. In embodiments, the NOAEL dose amount of S(−) pramipexole is an amount that does not exceed 1.5 mg, does not exceed 0.5 mg, or more preferably does not exceed 0.05 mg. In another preferred embodiment, the NOAEL dose amount of S(−) pramipexole is an amount that does not exceed 0.0007 mg/kg per unit dose.

The compositions of pramipexole may have a chiral purity for the R(+) enantiomer of at least 99.5%, preferably at least 99.6%, preferably at least 99.7%, preferably at least 99.8%, preferably at least 99.9%, preferably at least 99.5% and more preferably at least 99.99%. In a preferred embodiment, the chiral purity for the R(+) enantiomer of pramipexole, or pharmaceutically acceptable salt thereof, may be 100%. In embodiments, the composition may further comprise a pharmaceutically acceptable carrier. The therapeutically effective amount of R(+) pramipexole, or the pharmaceutically acceptable salt thereof, may be effective as an inhibitor of oxidative stress, an inhibitor of lipid peroxidation or in detoxification of oxygen radicals.

Embodiments of the invention include compositions that may be administered orally, preferably as a solid oral dose, and more preferably as a solid oral dose that may be a capsule or tablet. In preferred embodiments, the compositions of the present invention may be formulated as tablets for oral administration.

Another embodiment of the invention is a composition consisting essentially of a therapeutically effective amount of R(+) pramipexole and a non-effective dose amount of S(−) pramipexole. Another embodiment of the invention is a composition consisting essentially of a therapeutically effective amount of R(+) pramipexole and a NOAEL dose amount of S(−) pramipexole. Another embodiment of the invention is a composition consisting of a therapeutically effective amount of R(+) pramipexole and a non-effective dose amount of S(−) pramipexole. Such compositions may preferably be therapeutic or pharmaceutical compositions. Another embodiment of the invention is a composition consisting of a therapeutically effective amount of R(+) pramipexole and a NOAEL dose amount of S(−)pramipexole. Such compositions may preferably be therapeutic or pharmaceutical compositions.

Another embodiment of the invention is a pharmaceutical composition comprising a therapeutically effective amount of R(+) pramipexole and a non-effective dose amount of S(−) pramipexole administered in a unit dose form. Preferable unit dose forms include those suitable for oral administration, including but not limited to, capsules, tablets and the like. Table 1 shows various exemplary embodiments. Shown in each column of Table 1 is the amount of S(−) pramipexole that may be co-administered in a non-effective dose amount as a function of the chiral purity of the composition for the R(+) enantiomer of pramipexole. The therapeutically effective amount of R(+) pramipexole may preferably be about 50 mg to about 5,000 mg, preferably from about 100 mg to about 3,000 mg, preferably from about 300 mg to about 1,500 mg, or more preferably from about 500 mg to about 1,000 mg. This dose may be administered as a single daily dose, or may be divided into several doses administered throughout the day, for example, 1 to 5 doses per day. The non-effective dose amount of S(−) pramipexole may be preferably below 1.0 mg/day, more preferably below 0.5 mg/day, and more preferably below 0.125 mg/day. Thus, as a non-limiting example, a dose of 500 mg/day administered to a patient as a single unit dose may have a chiral purity for the R(+) enantiomer of pramipexole of at least about 99.80%, so that the non-effective dose amount of S(−) pramipexole may remain below 1.0 mg/day, more preferably about 99.90% so that the non-effective dose amount of S(−) parmipexole may remain below 0.5 mg/day, and more preferably about 99.75% so that the non-effective dose amount of S(−) pramipexole may remain below 0.125 mg/day. With reference to Table 1, any combination of chiral purity and unit dose may be used which allows for the desired combination of a therapeutically effective amount of R(+) pramipexole and a non-effective dose amount of S(−) pramipexole as stated herein.

A preferred embodiment of the invention is a pharmaceutical composition suitable for oral administration comprising an amount of R(+) pramipexole greater than 100 mg and a non-effective dose amount of S(−) pramipexole that is less than 0.125 mg. Another preferred embodiment is a pharmaceutical composition suitable for oral administration comprising an amount of R(+) pramipexole greater than 250 mg and a non-effective dose amount of S(−) pramipexole that is less than 0.125 mg. Yet another preferred embodiment of the invention is a pharmaceutical compositions suitable for oral administration comprising an amount of R(+) pramipexole greater than 500 mg and a non-effective dose amount of S(−) pramipexole that is less than 0.125 mg. Preferred pharmaceutical compositions for oral administration include tablets, capsules and the like.

Another embodiment of the invention is a pharmaceutical commotion formulated as a tablet suitable for oral administration comprising an amount of R(+) pramipexole greater than 50 mg and a non-effective dose amount of S(−) pramipexole that is less than 0.50 mg, preferably an amount of R(+) pramipexole greater than 100 mg and a non-effective dose amount of S(−) pramipexole that is less than 0.50 mg, and more preferably an amount of R(+) pramipexole greater than 250 mg and a none-effective dose amount of S(−) pramipexole that is less than 0.50 mg. Another preferred embodiment is a pharmaceutical composition formulated as a tablet suitable for oral administration comprising an amount of R(+) pramipexole greater than 500 mg and a non-effective dose amount of S(−) pramipexole that is less than 0.50 mg.

TABLE 1
Prefrrred non-effective dose amounts of S(−) pramipexole
based on the chiral purity of the composition for R(+) pramipexole

| Percent | | | | | | | | | | | |
|---|---|---|---|---|---|---|---|---|---|---|---|
| Chiral | Unit Dose Amount of R(+) pramipexole (mg) | | | | | | | | | | |
| Purity | 20 | 25 | 50 | 75 | 100 | 120 | 150 | 200 | 250 | 500 | 1000 |

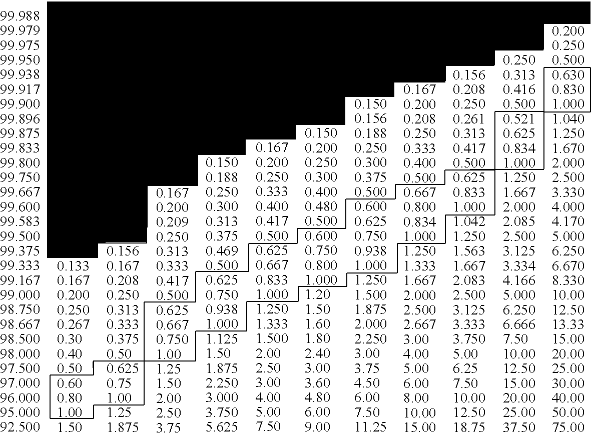

A preferred non-effective dose amount of the S(−) pramipexole may be below 1.0 mg: more preferably below 0.5 mg. and more preferably below 0.125 mg.

Another embodiment of the invention is a pharmaceutical composition formulated as a tablet suitable for oral administration comprising an amount of R(+) pramipexole greater than 50 mg and a non-effective dose amount of S(−) pramipexole that is less than 0.25 mg, preferably an amount of R(+) pramipexole greater than 100 mg and a non-effective dose amount of S(−) pramipexole that is less than 0.25 mg, and more preferably an amount of R(+) pramipexole greater than 250 mg and a non-effective dose amount of S(−) pramipexole that is less than 0.25 mg. Another preferred embodiment is a pharmaceutical composition formulated as a tablet suitable for oral administration comprising an amount of R(+) pramipexole greater than 500 mg and a non-effective dose amount of S(−) pramipexole that is less than 0.25 mg.

Another embodiment of the invention is a pharmaceutical composition comprising a therapeutically effective amount of R(+) pramipexole and a NOAEL dose amount of S(−) pramipexole administered in a unit dose form. Preferable unit dose forms include those suitable for oral administration, including but not limited to, capsules, tablets and the like. Table 2 shows various exemplary embodiments. Shown in each column of Table 2 is the amount of S(−) pramipexole that may be co-administered in NOAEL dose amount as a function of the chiral purity of the composition for the R(+) enantiomer of pramipexole. The therapeutically effective amount of R(+) pramipexole may preferably be about 50 mg to about 5,000 mg, preferably from about 100 mg to about 3,000 mg, preferably from about 300 mg to about 1,500 mg, more preferably from about 500 to about 1,000 mg. This dose may be administered as a single daily dose, or may be divided into several doses administered throughout the day, for example 1 to 5 doses per day. The NOAEL dose of S(−) pramipexole may be preferably below 1.5 mg, preferably below 0.5 mg, or more preferably below 0.05 mg. Thus, as a non-limiting example, an embodiment of the invention may be a dose of 1,500 mg/day administered to a patient as a single unit dose which may have a chiral purity for the R(+) enantiomer of pramipexole that is at least about 99.967% so that the non-adverse dose of S(−) pramipexole may remain below 0.50 mg/dose. Alternatively, a dose of 1,500 mg/day administered to a patient as three individual doses of 500 mg may have a chiral purity of the R(+) pramipexole that is at least about 99.90% so that the non-adverse dose of S(−) pramipexole may remain below 0.50 mg/dose or 1.5 mg/day. With reference to Table 2, any combination of chiral purity and unit dose may be used which allows for the desired combination of a therapeutically effective amount of R(+) pramipexole and a non-adverse effect dose amount of S(−) pramipexole as stated herein.

Another embodiment of the invention is a pharmaceutical composition formulated as a table suitable for oral administration comprising an amount of R(+) pramipexole greater than 50 mg and a NOAEL dose amount of S(−) pramipexole that is less than 0.05 mg, preferably an amount of R(+) pramipexole greater than 100 mg and a NOAEL dose amount of S(−) pramipexole that is less than 0.05 mg, and more preferably an amount of R(+) pramipexole greater than 250 and a NOAEL dose amount of S(−) pramipexole that is less than 0.05 mg. Another preferred embodiment is a pharmaceutical composition formulated as a tablet suitable for oral administration comprising an amount of R(+) pramipexole greater than 500 mg and a NOAEL dose amount of S(−) pramipexole that is less than 0.05 mg.

TABLE 2
Preferred no observable adverse effect level doses of S(−) pramipexole
based on the chiral purity of the composition for R(+) pramipexole

| Percent | | | | | | | | | | | | | |
|---|---|---|---|---|---|---|---|---|---|---|---|---|---|
| Chiral | Unit Dose Amount of R(+) pramipexole (mg) | | | | | | | | | | | | |
| Purity | 20 | 25 | 30 | 50 | 75 | 100 | 120 | 150 | 200 | 250 | 500 | 1000 | 1500 |

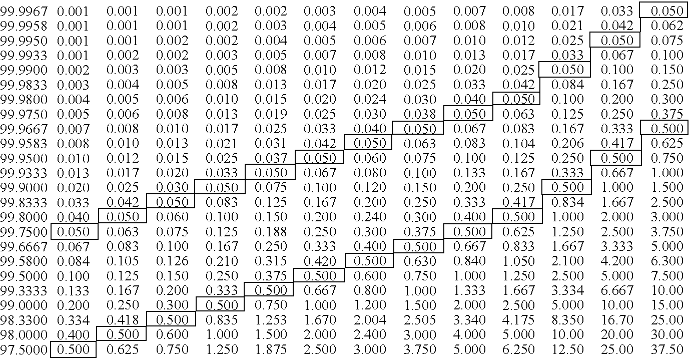

A preferred no observable adverse effect level (NOAEL) dose amount of the S(−) pramipexole may be below 0.5 mg, preferably below 0.05 mg.

The compounds of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, intravesicularly, by inhalation, by depot injections, or by implants. Thus, modes of administration for the compounds of the present invention (either alone or in combination with other pharmaceuticals) can be, but are not limited to, sublingually, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

The doses of the R(+) pramipexole which may be administered to a patient in need thereof may range between about 0.1 mg/kg per day and about 1,000 mg/kg per day. This dose may be administered as a single daily dose, or may be divided into several doses which are administered throughout the day, such as 1 to 5 doses. The route of administration may include oral, sublingually, transdermal, rectal, or any accessible parenteral route. One of ordinary skill in the art will understand and appreciate the dosages and timing of said dosages to be administered to a patient in need thereof. The doses and duration of treatment may vary, and may be base don assessment by one of ordinary skill in the art based on monitoring and measuring improvements in neuronal and non-neuronal tissues. This assessment may be made based on outward physical signs of improvement, such as increased muscle control, or on internal physiological signs or markers. The doses may also depend on the condition or disease being treated, the degree of the condition or disease being treated and further on the age and weight of the patient.

Specific modes of administration will depend on the indication. The selection of the specific route of administration and the dose regimen may be adjusted or titrated by the clinician according to methods known to the clinician in order to obtain the optimal clinical response. The amount of compound to be administered may be that amount which is therapeutically effective. The dosage to be administered may depend on the characteristics of the subject being treated, e.g., the particular animal or human subject treated, age, wiehgt, health, types of concurrent treatment, if any, and frequency of treatments, and can be easily determined by one of skill in the art (e.g., by the clinician).

A preferable route of administration of the compositions of the present invention may be oral, with a more preferable route being in the form of tablets, capsules, lozenges and the like. In preferred embodiments, the compositions of the present invention may be formulated as tablets for oral administration. A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powered compound moistened with an inert liquid diluent.

The tablets may be uncoated or they may be coated by known techniques, optionally to delay disintegration and absorption in the gastrointestinal tract and thereby providing a sustained action over a longer period. The coating may be adapted to release the active compound in a predetermined pattern (e.g., in order to achieve a controlled release formulation) or it may be adapted not to release the active compound until after passage of the stomach (enteric coating). The coating may be a sugar coating, a film coating (e.g., based on hydroxypropyl methylcellulose, methycellulose, methyl hydroxyethylcellulose, hydroxypropylcelluose, carboxymethylcelluose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl momostearate or glyceryl distearate may be employed. The solid table compositions may include a coating adapted to protect the composition from unwanted chemical changes, (e.g., chemical degradation prior to the release of the active drug substance).

Pharmaceutical formulations containing the compounds of the present invention and a suitable carrier may also be any number of solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's the Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well know in the art. As used herein, the term “pharmaceutically acceptable carrier” means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glcerin, sorbitol, mannitol and polyethylene glycol; esters such as ethyl oleate and ethyl laurate, agar; buffering agents such as magnesium hydroxide and aluminum hydroxide; alginic acid; progen-free water; isotonic saline, Ringer's solution; ethyl alcohol and phosphate buffer solutions, as well as other non-toxic compatible substances used in pharmaceutical formulations. Such carriers enable the compounds of the invention to be formulated as tablets, pills, dragees, capsules, liquids, gels, syrups, slurries, suspensions and the like, for oral ingestion by a patient to be created. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Formulations for oral use may also be presented as hard gelatin capsules wherein the active ingredient is mixed with an inert solid diluent, for example, calcium carbonate, calcium phosphate or kaolin, or as soft gelating capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gun acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

For buccal or sublingual admiistraitokn, the compositions can take the form of tablets, flash melts or lozenges formulated in any conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical and therapeutic compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

Various aspects of the present invention will be illustrated with reference to the following non-limiting examples.

### EXAMPLES

### Example 1

### Measurement of the Dopamine Receptor Affinities for the R(+) and S(−) Enantiomers of Pramipexole

The S(−) enantiomer of pramipexole has historically been characterized as a high affinity dopamine receptor ligand at the D₂ (both the S and L isoforms), D₃ and D₄ receptors, although the highest affinity is seen for the D₃ receptor subtype. The dopamine receptor ligand affinity of S(−) pramipexole from several clinical trials and journal publications has been tabulated (data is reproduced in Table 3). Although the conditions under which each study or experiment was carried out are slightly different, and different radio-ligands were used, the data show comparable affinities for the various dopamine receptors. Studies on the dopamine receptor affinity of the R(+) enantiomer of pramipexole are also shown in Table 3. These data demonstrate an unexpectedly large difference in the affinities of the two enantiomers of pramipexole for all dopamine receptors, with the R(+) enantiomer showing about 5,000-fold less affinity for the D₃ receptor subtype than the S(−) enantiomer, and a >10,000-fold lower affinity for the D_{2L} and D_{2S} receptor subtypes.

TABLE 3
Comparative human dopamine receptor
affinity for pramipexole enantiomers

| | |
|---|---|
| S(−) pramipexole * | R(+) pramipexole ** |

| Receptor | Kⱼ (nM) | Kⱼ (nM) | IC₅₀ (nM) |
|---|---|---|---|
| D₁ | >50,000 | >100,000 | >100,000 |
| D_{2S} | 2.2 | 29,000 | 87,000 |
| D_{2L} | 3.9 | >100,000 | >100,000 |
| D₃ | 0.5 | 2,700 | 12,000 |
| D₄ | 5,1 | 8,700 | 22,000 |
| D₅ | >50,000 | >100,000 | >100,000 |

* Historic data
** Data from the present studies.

The R(+) pramipexole was supplied as dry powder to the preclinical pharmacology service Cerep by the manufacturer AMRI. Solutions of R(+) pramipexole were prepared from stock solutions in DMSO. Eight concentrations were tested: 50 nM, 100 nM, 500 nM, 1 μM, 5 μM, 10 μM, 50 μM, 100 μM. These concentrations were tested in either CHO (Chinese hamster ovary) or HEK293 (human embryonic kidney) cell lines expressing human cloned dopamine receptors (D₁, D_{2S}, D_{2L}, D₃, D₄, D₅). The radio-ligand in each case was either [³H] spiperone or [³H] SCH23390 (a classic D₁ dopamine receptor antagonist R-(+)-7-Chloro-8-hydroxy-3-methyl-1-phenyl-2,3,4,5-tetrahydro-1H-3-benzazepine hydrochloride), both antagonists at 0.3 nM. Incubation was for 60 minutes, and data were collected for 2 repeats using scintillation counting. Group results for the interaction of R(+) pramipexole with each receptor are expressed as both IC₅₀ and Kᵢ in Table 3.

These data indicate that Kᵢ values of pramipexole for these receptors are larger by a factor of at from at least 1000 to greater than 10,000 for the R(+) enantiomer when compared to historic literature values for the S(−) enantiomer. These data also suggest that if dopamine receptor affinity is the major contributing factor to limiting dose tolerance of the S(−) enantiomer, then pure preparations of the R(+) enantiomer should have a maximum tolerated dose (MTD) and/or a no observable adverse effect level dose (NOAEL) of at least 1000 greater than the S(−) enantiomer's MTD and/or NOAEL. Thus, even a small contamination of the R(+) pramipexole compositions of the present invention by the S(−) enantiomer, at levels as low as 0.5% or less, may effect the observed MTD and NOAEL.

### Example 2

### In vivo Studies to Determine the MTD and NOAEL in Dogs for 100% Pure Preparations of the R(+) and S(−) Enantiomers of Pramipexole, and a Pramipexole Mixture (R 99.5%/S 0.5%)

The following in vivo study in beagle dogs was undertaken to test the hypothesis that the large observed difference in receptor binding affinities for the R(+) and S(−) enantiomers of pramipexole will translate to a large observed difference in the observed maximum tolerated dose (MTD) and/or no observable adverse effect level (NOAEL) of the two enantiomers. Dogs were administered preparations of each enantiomer prepared as a highly purified compound (100% pure preparations (within the limits of analytical detectability)), or a preparation of the pramipexole containing 99.5% of the R(+) enantiomer mixed with 0.5% of the S(−) enantiomer.

Three groups of four non-naïve beagle dogs, were used in the study. Each group was administered various doses of either the R(+) or S(−) enantiomer prepared as a highly purified compound, or a preparation of the pramipexole mixture containing 99.5% of the R(+) enantiomer and 0.5% of the S(−) enantiomer. Doses were administered orally by gavage and clinical observations were taken continuously following dosing: hourly for the first four hours, and then twice daily cage-side observations for the duration of the inter-dose of post-dose interval. Observations were made of clinical signs, mortality, injury and availability of food and water. Animals were fasted for 24 hr prior to dosing. Dogs in each group were exposed to only one of the purified pramipexole enantiomers or to the pramipexole moisture; each dose was administered only once, with a subsequent dose administered after a recovery period of 4 days. The data are summarized in Table 4.

A NOAEL was established at a dose level of 25 mg/kg for the R(+) enantiomer when administered to non-naïve dogs, while a dose level of 75 mg/kg may be considered an MTD in non-naïve dogs. For the S(−) enantiomer, a NOAEL of 0.00125 mg/kg and an MTD of 0.0075 mg/kg was found. For the composition containing a mixture of the two enantiomers (99.5% R(+) pramipexole and 0.5% S(−) pramipexole), the NOAEL was found to be 0.25 mg/kg, which corresponds to a dose of 0.00125 mg/kg of the S(−) enantiomer, while the MTD is 1.5 mg/kg, which corresponds to a dose of 0.0075 mg/kg of the S(−) enantiomer. These data indicate that the NOAEL for the R(+) enantiomer of pramipexole is approximately 20,000-fold greater than for the S(−) enantiomer in non-naïve dogs, while the MTD is about 10,000-fold greater.

TABLE 4
Clinical observations in male beagle dogs for administration of pramipexole compositions
SUMMARY OF CLINICAL FINDINGS*

| | Dose Amount (mg/kg) | | | | | | | | |
|---|---|---|---|---|---|---|---|---|---|
| | 7.5 | 25 | 75 | 0.0075 | 0.025 | 0.00125 | 1.5 | 5 | 0.25 |
| | R(+) | R(+) | R(+) | S(−) | S(−) | S(−) | mixture** | mixture | mixture |
| | (Day 1) | (Day 4) | (Day 8) | (Day 1) | (Day 4) | Day 8) | (Day 1) | (Day 4) | (Day 8) |
| Behavior/Activity | | | | | | | | | |
| Activity decreased | 0/4 | 0/4 | 2/4 | 3/4 | 4/4 | 0/4 | 4/4 | 4/4 | 0/4 |
| Convulsions - clonic | 0/4 | 0/4 | 1/4 | 0/4 | 0/4 | 0/4 | 0/4 | 0/4 | 0/4 |
| Salivation | 0/4 | 0/4 | 3/4 | 0/4 | 0/4 | 0/4 | 0/4 | 0/4 | 0/4 |
| Tremors | 0/4 | 0/4 | 4/4 | 1/4 | 3/4 | 0/4 | 1/4 | 2/4 | 0/4 |
| Excretion | | | | | | | | | |
| Emesis | 0/4 | 0/4 | 2/4 | 3/4 | 4/4 | 0/4 | 1/4 | 3/4 | 1/4 |
| Feces hard | 1/4 | 0/4 | 0/4 | 1/4 | 0/4 | 0/4 | 0/4 | 0/4 | 0/4 |
| Feces mucoid | 0/4 | 0/4 | 0/4 | 0/4 | 0/4 | 0/4 | 1/4 | 1/4 | 0/4 |
| Feces soft | 0/4 | 0/4 | 1/4 | 0/4 | 0/4 | 0/4 | 2/4 | 1/4 | 1/4 |
| Feces watery | 0/4 | 0/4 | 0/4 | 0/4 | 0/4 | 0/4 | 1/4 | 1/4 | 0/4 |
| External Appearance | | | | | | | | | |
| Lacrimation | 0/4 | 0/4 | 0/4 | 0/4 | 0/4 | 0/4 | 0/4 | 0/4 | 0/4 |
| Eye/Ocular | | | | | | | | | |
| Pupils dilated | 0/4 | 0/4 | 2/4 | 0/4 | 0/4 | 0/4 | 0/4 | 0/4 | 0/4 |
| Pelage/Skin | | | | | | | | | |
| Skin warm to touch | 1/4 | 0/4 | 1/4 | 0/4 | 0/4 | 0/4 | 0/4 | 0/4 | 0/4 |

*Number of animals affected/Total number of animals
**Mixture of 99.5% R(+) pramipexole and 0.5% S(−) pramipexole.

The data shown in Table 4 indicate that the dopamine receptor affinities identified (see Table 3) contribute in a straightforward fashion to the observed differences in the MTD and NOAEL doses for the R(+) and S(−) enantiomers of pramipexole. These data also indicate that the chiral purity for the R(+) enantiomer of pramipexole in embodiments of the compositions of the present invention (refer to Tables 1 and 2) may need to be in excess of 99.9%, depending on the final total dose, to avoid the adverse side effects of S(−) pramipexole.

Further, the data in Table 4 demonstrate that the NOAEL and MTD for the combination composition (99.5% R(+) pramipexole and 0.5% S(−) pramipexole) may be determined directly by the dose of the S(−) enantiomer in the composition. Thus, a small (fractional percentage) contamination of a composition of R(+) pramipexole by the S(−) enantiomer may reduce the MTD and NOAEL of the composition. For example, in these experiments, the MTD of pramipexole was reduced from 75 mg/kg for the R(+) enantiomer to a total dose of 1.5 mg/kg of the mixed composition (a factor of 50), and the NOAEL was reduced from 25 mg/kg to 0.25 mg/kg, respectively (a factor of 100). Since the shift in MTD and NOAEL may be predicated by the dose of the S(−) enantiomer of pramipexole in the mixture, the shift for any unknown mixture may be calculated based on the percentage contamination of the R(+) pramipexole by the S(−) enantiomer, relative to the MTD and NOAEL, for S(−) pramipexole. This indicates that any contamination of an R(+) pramipexole dosing solution with S(−) pramipexole will have a measurable effect on these indicators of dose tolerability.

Although the present invention has ben described in considerable detail with reference to certain preferred embodiments thereof, other versions are possible. There the spirit and scope of the appended claims should not be limited to the description and the preferred versions contained within this specification.

1. A method of treating age-related macular degeneration comprising administering a therapeutically effective amount of R(+) pramipexole.

2. The method of claim 1, wherein said therapeutically effective amount of R(+) pramipexole is administered in a pharmaceutical composition.

3. The method of claim 2, wherein said pharmaceutical composition has a chiral purity for the R(+) enantiomer of pramipexole of 80% or greater.

4. The method of claim 2, wherein said pharmaceutical composition has a chiral purity for the R(+) enantiomer of pramipexole of 90% or greater.

5. The method of claim 2, wherein said pharmaceutical composition has a chiral purity for the R(+) enantiomer of pramipexole of 95% or greater.

6. The method of claim 2, wherein said pharmaceutical composition has a chiral purity for the R(+) enantiomer of pramipexole of 99% or greater.

7. The method of claim 1, wherein said the therapeutically effective amount of R(+) pramipexole is from about 50 milligrams to about 5000 milligrams.

8. The method of claim 1, wherein the therapeutically effective amount of R(+) pramipexole is from about 100 milligrams to about 3000 milligrams.

9. The method of claim 1, wherein the therapeutically effective amount of R(+) pramipexole is from about 300 milligrams to about 1500 milligrams.

10. The method of claim 1, wherein the therapeutically effective amount of R(+) pramipexole is from about 500 milligrams to about 1000 milligrams.

11. The method of claim 2, wherein said pharmaceutical composition is suitable for oral administration.

12. The method of claim 2, wherein said pharmaceutical composition is a solid oral dosage form.

13. The method of claim 2, wherein said pharmaceutical composition is a tablet.

14. The method of claim 2, wherein said pharmaceutical composition is a capsule.

15. The method of claim 2, wherein said pharmaceutical composition is a suitable for ocular administration.

16. The method of claim 2, wherein said pharmaceutical composition further comprises S(−) pramipexole in an amount that does not provide significant dopamine agonist activity.

17. The method of claim 2, wherein said pharmaceutical composition consists essentially of R(+) pramipexole.

18. The method of claim 2, wherein said pharmaceutical compositions further comprises an agent useful in treating age-related macular degeneration.

19. A method of treating of treating type II diabetes comprising administering a therapeutically effective amount of R(+) pramipexole.

20. The method of claim 19, wherein said therapeutically effective amount of R(+) pramipexole is administered in a pharmaceutical composition.

21. The method of claim 20, wherein said pharmaceutical composition has a chiral purity for the R(+) enantiomer of pramipexole of 80% or greater.

22. The method of claim 20, wherein said pharmaceutical composition has a chiral purity for the R(+) enantiomer of pramipexole of 90% or greater.

23. The method of claim 20, wherein said pharmaceutical composition has a chiral purity for the R(+) enantiomer of pramipexole of 95% or greater.

24. The method of claim 20, wherein said pharmaceutical composition has a chiral purity for the R(+) enantiomer of pramipexole of 99% or greater.

25. The method of claim 19, wherein said the therapeutically effective amount of R(+) pramipexole is from about 50 milligrams to about 5000 milligrams.

26. The method of claim 19, wherein the therapeutically effective amount of R(+) pramipexole is from about 100 milligrams to about 3000 milligrams.

27. The method of claim 19, wherein the therapeutically effective amount of R(+) pramipexole is from about 300 milligrams to about 1500 milligrams.

28. The method of claim 19, wherein the therapeutically effective amount of R(+) pramipexole is from about 500 milligrams to about 1000 milligrams.

29. The method of claim 20, wherein said pharmaceutical composition is suitable for oral administration.

30. The method of claim 20, wherein said pharmaceutical composition is a solid oral dosage form.

31. The method of claim 20, wherein said pharmaceutical composition is a tablet.

32. The method of claim 20, wherein said pharmaceutical composition is a capsule.

33. The method of claim 20, wherein said pharmaceutical composition further comprises S(−) pramipexole in an amount that does not provide significant dopamine agonist activity.

34. The method of claim 20, wherein said pharmaceutical composition consists essentially of R(+) pramipexole.

35. The method of claim 20, wherein said pharmaceutical composition further comprises an agent useful in treating type II diabetes.

36. A method of treating of treating skin disorders comprising administering a therapeutically effective amount of R(+) pramipexole.

37. The method of claim 36, wherein said therapeutically effective amount of R(+) pramipexole is administered in a pharmaceutical composition.

38. The method of claim 37, wherein said pharmaceutical composition has a chiral purity of the R(+) enantiomer of pramipexole of 80% or greater.

39. The method of claim 37, wherein said pharmaceutical composition has a chiral purity for the R(+) enantiomer of pramipexole of 90% or greater.

40. The method of claim 37, wherein said pharmaceutical composition has a chiral purity for the R(+) enantiomer of pramipexole of 95% or grater.

41. The method of claim 37, wherein said pharmaceutical composition has a chiral purity for the R(+) enantiomer of pramipexole of 99% or greater.

42. The method of claim 36, wherein said the therapeutically effective amount of R(+) pramipexole is from about 50 milligrams to about 5000 milligrams.

43. The method of claim 36, wherein the therapeutically effective amount of R(+) pramipexole is from about 100 milligrams to about 3000 milligrams.

44. The method of claim 36, wherein the therapeutically effective amount of R(+) pramipexole is from about 300 millgrams to about 1500 milligrams.

45. The method of claim 36, wherein the therapeutically effective amount of R(+) pramipexole is from about 500 milligrams to about 1000 milligrams.

46. The method of claim 37, wherein said pharmaceutical composition is suitable for oral administration.

47. The method of claim 37, wherein said pharmaceutical composition is a solid oral dosage form.

48. The method of claim 37, wherein said pharmaceutical composition is a tablet.

49. The method of claim 37, wherein said pharmaceutical competition is a capsule.

50. The method of claim 37, wherein said pharmaceutical composition is suitable for topical administration.

51. The method of claim 37, wherein said pharmaceutical composition further comprises S(−) pramipexole in an amount that does not provide significant dopamine agonist activity.

52. The method of claim 37, wherein said pharmaceutical consists essentially of R(+) pramipexole.

53. The method of claim 37, wherein the pharmaceutical compositions further comprises an agent useful in treating skin disorders.

54. A method of treating of treating cardiovascular disorders comprising administering a therapeutically effective amount of R(+) pramipexole.

55. The method of claim 54, wherein said therapeutically effective amount of R(+) pramipexole is administered in a pharmaceutical composition.

56. The method of claim 55, wherein said pharmaceutical composition has a chiral purity for the R(+) enantiomer of pramipexole of 80% or greater.

57. The method of claim 55, wherein said pharmaceutical composition has a chiral purity for the R(+) enantiomer of pramipexole of 90% or greater.

58. The method of claim 55, wherein said pharmaceutical composition has a chiral purity for the R(+) enantiomer of pramipexole of 95% or greater.

59. The method of claim 55, wherein said pharmaceutical composition has a chiral purity for the R(+) enantiomer of pramipexole of 99% or greater.

60. The method of claim 54, wherein said the therapeutically effective amount of R(+) pramipexole is from about 50 milligrams to about 5000 milligrams.

61. The method of claim 54, wherein the therapeutically effective amount of R(+) pramipexole is from about 100 milligrams to about 3000 milligrams.

62. The method of claim 54, wherein the therapeutically effective amount of R(+) pramipexole is from about 300 milligrams to about 1500 milligrams.

63. The method of claim 54, wherein the therapeutically effective amount of R(+) pramipexole is from about 500 milligrams to about 1000 milligrams.

64. The method of claim 55, wherein said pharmaceutical composition is suitable for oral administration.

65. The method of claim 55, wherein said pharmaceutical composition is a solid oral dosage form.

66. The method of claim 55, wherein said pharmaceutical composition is a tablet.

67. The method of claim 55, wherein said pharmaceutical composition is a capsule.

68. The method of claim 55, wherein said pharmaceutical composition further comprises S(−) pramipexole in an amount that does not provide significant dopamine agonist activity.

69. The method of claim 55, wherein said pharmaceutical composition consists essentially of R(+) pramipexole.

70. The method of claim 55, wherein the pharmaceutical compositions further comprises an agent useful in treating cardiovascular disorders.

71. A method of treating of treating inflammatory disorders comprising administering a therapeutically effective amount of R(+) pramipexole.

72. The method of claim 71, wherein said therapeutically effective amount of R(+) pramipexole is administered in a pharmaceutical composition.

73. The method of claim 72, wherein said pharmaceutical composition has a chiral purity for the R(+) enantiomer of pramipexole of 80% or greater.

74. The method of claim 72, wherein said pharmaceutical composition has a chiral purity for the R(+) enantiomer of pramipexole of 90% or greater.

75. The method of claim 72, wherein said pharmaceutical composition has a chiral purity for the R(+) enantiomer of pramipexole of 95% or greater.

76. The method of claim 72, wherein said pharmaceutical composition has a chiral purity for the R(+) enantiomer of pramipexole of 99% or greater.

77. The method of claim 71, wherein said the therapeutically effective amount of R(+) pramipexole is from about 50 milligrams to about 5000 milligrams.

78. The method of claim 71, wherein the therapeutically effective amount of R(+) pramipexole is from about 100 milligrams to about 3000 milligrams.

79. The method of claim 71, wherein the therapeutically effective amount of R(+) pramipexole is from about 300 milligrams to about 1500 milligrams.

80. The method of claim 71, wherein the therapeutically effective amount of R(+) pramipexole is from about 500 milligrams to about 1000 milligrams.

81. The method of claim 72, wherein said pharmaceutical composition is suitable for oral administration.

82. The method of claim 72, wherein said pharmaceutical composition is a solid oral dosage form.

83. The method of claim 72, wherein said pharmaceutical composition is a tablet.

84. The method of claim 72, wherein said pharmaceutical composition further comprises S(−) pramipexole in an amount that does not provide significant dopamine agonist activity.

85. The method of claim 72, wherein said pharmaceutical composition consists essentially of R(+) pramipexole.

86. The method of claim 72, wherein the pharmaceutical compositions further comprises an agent useful in treating inflammatory disorders.
